(19) United States
(12) Patent Application Publication
(10) Pub. No.: US 2008/0243076 A1
(43) Pub. Date: Oct. 2, 2008

(54) Method and Apparatus For Venipuncture Site Location

(75) Inventors: Amir Hossein Goldan, Vancouver (CA); Ameneh Atai, West Vancouver (CA); Balraj Mattu, Surrey (CA); Ida Khodami, Vancouver (CA)

Correspondence Address: OYEN, WIGGS, GREEN & MUTALA LLP;480 - THE STATION, 601 WEST CORDOVA STREET, VANCOUVER, BC, V6B 1G1, CA

(73) Assignee: Simon Fraser University

(21) Appl. No.: 11/995,513

(22) PCT Filed: Jul. 11, 2006
(86) PCT No.: PCT/CA06/01108

§ 371 Date: Jan. 11, 2008

Related U.S. Application Data
(60) Provisional application No. 60/697,558, filed on Jul. 11, 2005.

(51) Int. Cl. A61M 5/42

(52) U.S. Cl. 604/116

(57) ABSTRACT

A vein locator comprises an energy source for directing incident energy toward organism tissue. At least a portion of the incident energy is reflected from structures within the organism tissue as reflected energy. The vein locator comprises an energy detector connected to detect energy reflected from the organism tissue. The incident energy is moved across a scan region a first time and the energy detector produces a corresponding first signal representative of at least one characteristic of the first reflected energy. A controller receives the first signal, identifies an extremum in the first signal and determines a threshold level based on the identified extremum. The incident energy is then moved across the scan region a second time and the energy detector produces a corresponding second signal representative of at least one characteristic of the second reflected energy. The controller compares the second signal to the threshold level to determine whether a current location of the incident beam represents a location of a vein.

### RELATED APPLICATIONS

This application claims the benefit of the filing date of U.S. application No. 60/697,558 filed 11 Jul. 2005 which is hereby incorporated herein by reference.

### TECHNICAL FIELD

This invention relates to locating veins within the tissue of living organisms. Particular embodiments of the invention provide methods and apparatus for locating venipuncture sites.

### BACKGROUND

Puncturing the veins of living organisms is an important part of the delivery of medical and/or veterinary services. Puncturing veins can be desirable to draw blood (e.g. for diagnostic purposes), to provide intravenous hydration and/or nutrition, to introduce fluids which aid in diagnostic procedures of various ailments (e.g. radiopaque dye) and to administer medicine, for example.

Through venipuncture is a common practice, difficulties can arise due to misjudgments in the location of veins. Locating and/or puncturing veins can be difficult in patients with certain physical conditions. For example, locating and/or puncturing veins can be difficult for obese people, people having scarred or dark pigmented epidermis, people having small veins (e.g. babies), people having veins that are frequently punctured (e.g. intravenous drug users), people with low blood pressure and/or people undergoing treatment for diseases (e.g. HIV, cancer and/or liver disease). In some circumstances, such as medical emergencies or when venipuncture is being performed by inexperienced individuals, locating and/or puncturing veins may be difficult even though the patients do not have chronic physical conditions which might otherwise impair venipuncture. Locating and/or puncturing the veins of animals can also be difficult (e.g. where the veins are located well below the skin, where the animals' skin is thick and/or opaque and where the animals are covered in fur).

Where locating and/or puncturing the veins of a patient is difficult, multiple needle punctures may be required to obtain an acceptable venipuncture site. Multiple punctures are not only painful, but can also cause bruising, nausea and infection.

There is a general desire among health care practitioners and patients for a system that aids in conducting venipuncture by locating veins and/or locating suitable venipuncture sites.

### SUMMARY

One aspect of the invention provides a vein locator. The vein locator comprises a transmitter for directing an incident energy beam toward organism tissue, such that at least a portion of the incident energy beam reflects from structures within the organism tissue as a reflected energy beam. The vein locator also comprises a receiver, which is connected to detect a first reflected energy beam when the incident energy beam is moved across a scan region a first time and configured to produce a corresponding first signal representative of at least one characteristic of the first reflected energy beam. The receiver is also connected to detect a second reflected energy beam when the incident energy beam is moved across the scan region a second time and configured to produce a corresponding second signal representative of at least one characteristic of the second reflected energy beam. The vein locator also comprises a controller connected to receive the first and second signals from the receiver and configured to: identify an extremum of the first signal and determine a threshold level based on the identified extremum; and compare the second signal to the threshold level while the incident energy beam is being moved across the scan region the second time to locate a vein.

Another aspect of the invention provides a method for locating a vein within organism tissue. The method comprises directing an incident energy beam toward the organism tissue. At least a portion of the incident energy beam reflects from structures with the organism tissue as a reflected energy beam. The method comprises moving the incident energy beam across a scan region of the organism tissue a first time and detecting a corresponding first reflected energy beam, thereby generating a first signal representative of at least one characteristic of the first reflected energy beam. An extremum of the first signal is identified and a threshold level is determined based on the identified extremum. The method comprises moving the incident energy beam across the scan region a second time and detecting a corresponding second reflected energy beam, thereby generating a second signal representative of at least one characteristic of the second reflected energy beam. While moving the incident energy beam across the scan region the second time, a vein is located on the basis of comparing the second signal to the threshold level.

Further aspects of the invention, further features of specific embodiments of the invention and applications of the invention are described below.

### BRIEF DESCRIPTION OF DRAWINGS

In drawings which show non-limiting embodiments of the invention:

FIG. 1 is a block diagram showing the components of a venipuncture site locator according to a particular embodiment of the invention;

FIG. 2 is a schematic block diagram illustrating a method for operating the FIG. 1 site locator according to a particular embodiment of the invention;

FIG. 3A is a graphic depiction of data collected in a representative example of the FIG. 2 scan mode in accordance with a particular embodiment of the invention;

FIG. 3B is a graphic depiction of data collected in a representative example of the FIG. 2 indicator mode in accordance with a particular embodiment of the invention;

FIG. 4 is a block diagram showing the components of a venipuncture site locator according to another particular embodiment of the invention;

FIG. 5 is a circuit diagram of a receiver suitable for use with the FIG. 4 site locator;

FIGS. 6A and 6B respectively depict waveforms representing receiver output signals in the vicinity of soft tissue and in the vicinity of a vein;

FIG. 7 is a schematic block diagram illustrating a method for operating the FIG. 4 site locator according to a particular embodiment of the invention;

FIG. 8 is a schematic block diagram of an amplitude detection process suitable for use in the FIG. 7 method; and

FIG. 9 shows a portion of a housing for the FIG. 4 site locator according to a particular embodiment of the invention.

### DESCRIPTION

Throughout the following description, specific details are set forth in order to provide a more thorough understanding of the invention. However, the invention may be practiced without these particulars. In other instances, well known elements have nor been shown or described in detail to avoid unnecessarily obscuring the disclosure. Accordingly, the specification and drawings are to be regarded in an illustrative, rather than a restrictive, sense.

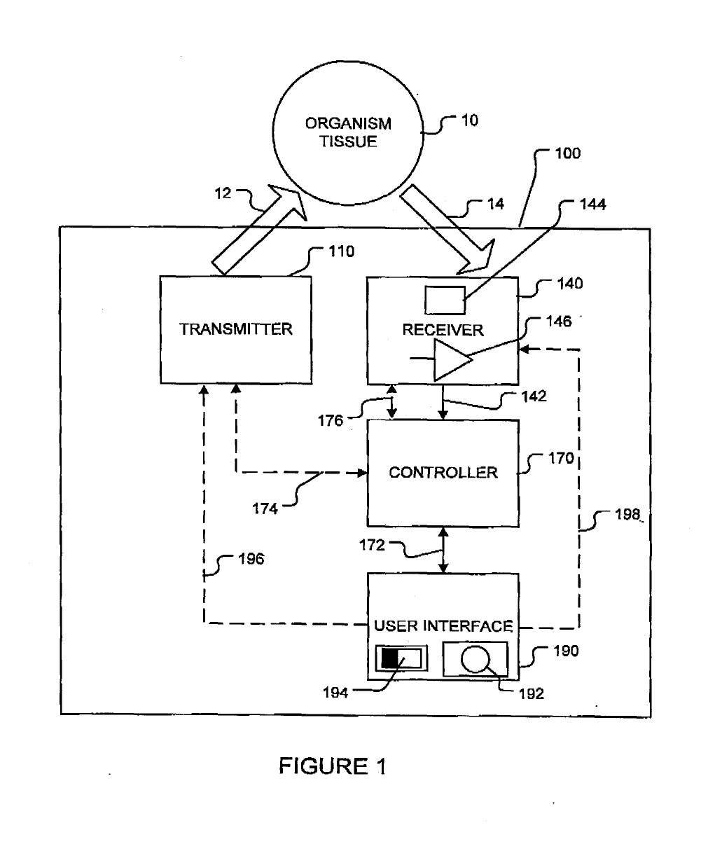

FIG. 1 schematically depicts a venipuncture site locator 100 according to a particular embodiment of the invention. Site locator 100 comprises an energy transmitter 110 which transmits a beam of energy 12 toward organism tissue 10. Organism tissue 10 may be the tissue of a human or animal for example and may comprise one or more veins and/or other structures (not shown in FIG. 1). Typically, such veins are located below the external surface of organism tissue 10. At least a portion of incident energy beam 12 penetrates the external surface of organism tissue 10 and reflects (as reflected beam 14) from the structures contained within organism tissue 10. The stress within organism tissue 10 interact differently with incident energy beam 12. Consequently, the characteristics of reflected beam 14 (e.g. the amount of reflected energy, the beam intensity, and/or the beam flux) depend on the nature of the structures within organism tissue 10. In one particular embodiment, transmitter 110 comprises a source of infrared or near infrared electromagnetic radiation which forms incident energy beam 12. At this wavelength the reflectance of blood may be significantly different than other soft tissue or hard tissue that may be located in organism tissue 10.

Site locator 100 also comprises an energy receiver 140 which detects one or more characteristics of reflected beam 14 and outputs an output signal 142 representative of such detected characteristic(s). Energy receiver 140 may comprise one or more transducers or sensors 144 operative to detect one or more characteristics of reflected beam 14 and convert these characteristic(s) of reflected beam 14 into one or more corresponding electrical signals. Non-limiting examples of characteristic(s) of reflected beam 14 that may be detected by receiver 140 include an amount of reflected energy, an intensity of reflected beam 14, a flux of reflected beam 14, a frequency of reflected beam 14 and a phase of reflected beam 14. Energy receiver 140 may also optionally comprise suitable signal conditioning circuitry 146, such as amplifiers, filters, demodulation circuitry and analog-to-digital (A/D) converters, for preliminary processing of the electrical signal(s) generated by sensor(s) 144. In some embodiments, some or all of signal conditioning circuitry 146 may be provided by controller 170.

Receiver 140 provides receiver output signal 142 to a controller 170. Controller 170 processes receiver output signal 142 to determine the location of one or more veins within organism tissue 10. Controller 170 is preferably programmed to execute suitable software instructions. Controller 170 may comprise, without limitation, a microprocessor, a computer-on-a-chip, the CPU of a computer or any other suitable microcontroller. Controller 170 may comprise a group of data processors. In some embodiments, receiver output signal 142 provided to controller 170 is an analog signal. In such embodiments, controller 170 may comprise an A/D converter (not shown in FIG. 1) which digitizes receiver output signal 142 to form a digital signal. In other embodiments, receiver output signal 142 is a digital signal which has been digitized by signal conditioning circuitry 146 of receiver 140 prior to being received at controller 170. In some embodiments, transmitter modulates incident beam 12 at a carrier frequency. In such embodiments, receiver 140 and/or controller 170 may comprise digital or analog demodulation components (not shown in FIG. 1).

In addition to receiving and processing receiver output signal 142, controller 170 may optionally output a signal 174 for controlling transmitter 110 and/or a signal 176 for controlling receiver 140. For example, signal 174 may be used by controller 170 to turn incident beam 12 on and off and/or to control one or more other characteristics of indent beam 12. Similarly, controller 170 may use signal 176 to turn sensor(s) 144 on or off and/or to adjust the parameters of signal conditioning circuitry 146.

Site locator 100 also comprises a user interface 190. In the illustrated embodiment, user interface 190 comprises one or more output components 192. Output component 192 may be responsive to a signal 172 received from controller 170. Output component 192 may provide information to users about the structures in organism tissue 10. In some embodiments, output component 192 indicates the location(s) of vein(s) suitable for venipuncture. In one particular embodiment, output component 192 comprises a LED (or some other visible device) which activates in response to signal 172 when site locator 100 is located at a suitable venipuncture site. Additionally or alternatively, output component 192 may comprise a marking device (e.g. a surgical marker or an indentation marker that leaves a visible indentation on the skin), which marks the skin of the patient in response to signal 172 at the location of a suitable venipuncture site. Output component 192 may comprise other forms of output devices, including, without limitation, an audible indicator, a LCD display or the like.

User interface 190 may also comprise one or more input components 194. Input component 194 allows users to control the operation of site locator 100. In some embodiments, input component 194 allows users to provide operational information to controller 170 via signal 172 and controller 170 in turn controls the operation of site locator in response to this operational information. In other embodiments, input component 194 additionally or alternatively allows users to directly control the operation of transmitter 110 via optional signal 196 and/or the operation of receiver 140 via optional signal 198. In one particular embodiment, input component 194 comprises a switch which tams site locator 100 on or off and a switch which changes site locator 100 between its scan mode operation and its indicator mode operation (see further description of scan mode and indicator mode below).

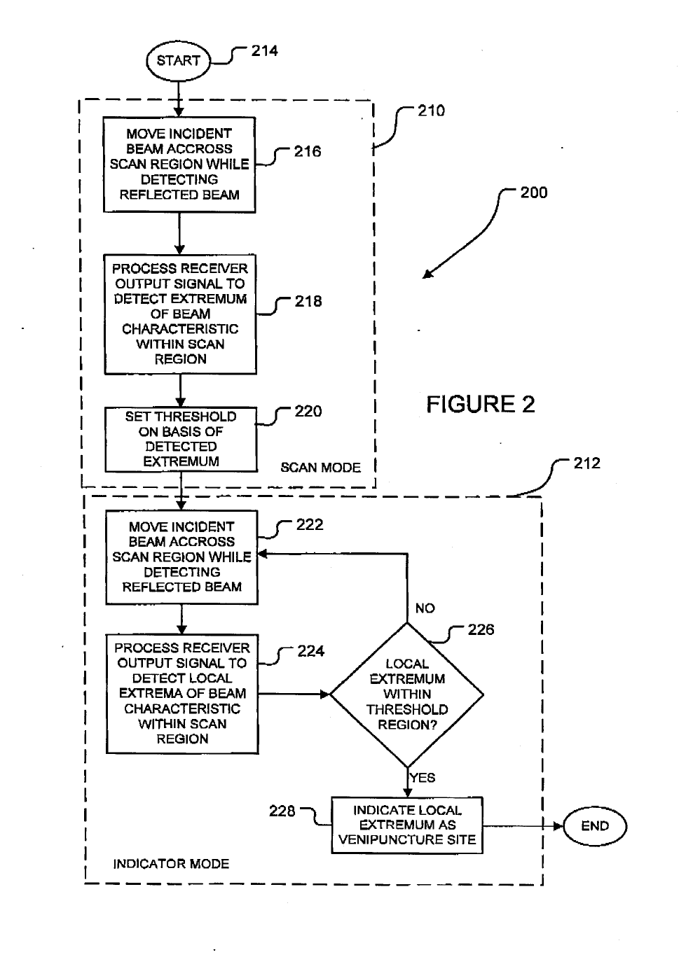

FIG. 2 depicts a method 200 for operating site locator 100 according to a particular embodiment of the invention. In the illustrated embodiment, method 200 comprises a scan mode 210 and an indicator mode 212. In general, scan mode 210 involves calibrating site locator 100 to a particular scan region on a patient and indicator mode 212 indicates the location(s) of vein(s) within the scan region. A scan region may generally comprise any region of the patient's body where it may be desired to locate and/or puncture a vein. As a part of scan mode 210, incident beam 12 is moved across the scan region and controller 170 establishes a threshold level Qₜₕᵣₑₛₕ for the particular scan region. The threshold level Qₜₕᵣₑₛₕ may comprise an upper threshold and/or a lower threshold. In indicator mode 212, incident beam 12 is again moved across the scan region and controller 170 compares receiver output signal 142 to the threshold level Qₜₕᵣₑₛₕ. A receiver output signal 142 greater than threshold level Qₜₕᵣₑₛₕ (in the case where Qₜₕᵣₑₛₕ is an upper threshold) or less than threshold level Qₜₕᵣₑₛₕ (in the case where Qₜₕᵣₑₛₕ is a lower threshold) is indicative of the location of a vein. Such vein location(s) may be suitable for venipuncture.

Method 200 begins in block 214 where site locator 100 is turned on and transmitter 110 outputs incident energy beam 12. Method 200 then enters scan mode 210 and proceeds to block 216. In block 216, incident beam 12 is caused to move across a scan region. Preferably, incident beam 12 moves from a first edge of the scan region across the scan region to an opposing edge of the scan region. In some embodiments, site locator 100 is physically moved across the scan region to effect the block 216 movement of incident beam 12. In other embodiments, transmitter 110 comprises one or more adjustable beam manipulation elements (not shown), which allow transmitter 110 to effect the block 216 movement of incident beam 12 without moving site locator 100. Such beam manipulation elements may include moveable optical elements, for example. The reflection of incident beam 12 from the structures within the scan region creates a reflected beam 14. As incident beam 12 moves across the scan region in block 216, receiver 140 detects reflected beam 14.

Receiver 140 generates receiver output signal 142 in response to detecting reflected beam 14 and passes receiver output signal 142 to controller 170. As discussed above, receiver output signal 142 is representative of one or more characteristics of reflected beam 14. In block 218, controller 170 processes receiver output signal 142 to detect an extremum within the scan region. In one particular embodiment, receiver 140 detects the intensity of reflected beam 14 and, as a part of block 218, controller 170 processes receiver output signal 142 to detect a minimum intensity of reflected beam 14 within the scan region. Such a minimum intensity level may be representative of the relatively low reflectance of blood (i.e. a vein) when compared to the higher reflectance of soft tissue and hard tissue within the scan region.

In currently preferred embodiments, controller 170 commences the extremum detection process of block 218 in “real time” as soon as it begins receiving receiver output signal 142 generated in block 216. In other embodiments, controller 170 may wait until block 216 is partially completed or until the conclusion of block 216 prior to commencing the extremum detection procedures of block 218. In such embodiments, controller 170 may have access to a memory buffer for storing receiver output signal 142 (or samples thereof). At the conclusion of block 218, controller 170 has detected an extremum of receiver output signal 142 within the scan region.

Method 200 the proceeds to block 220, where controller 170 uses the extremum determined in block 218 to set a threshold level Qₜₕᵣₑₛₕ. In embodiments where the block 218 extremum is a maximum Qₘₐₓ, the block 220 threshold level Qₜₕᵣₑₛₕ is less than the block 218 maximum Qₘₐₓ. Conversely, in embodiments where the block 218 extremum is a minimum Qₘᵢₙ, the block 220 threshold level Qₜₕᵣₑₛₕ is greater than the block 218 minim Qₘᵢₙ. In some embodiments, controller 170 determines the block 220 threshold level Qₜₕᵣₑₛₕ by offsetting the extremum determined in block 218. In other embodiments, controller 170 determines the block 220 threshold level Qₜₕᵣₑₛₕ by scaling the extremum determined in block 218. In still other embodiments, controller 170 determines the block 220 threshold level Qₜₕᵣₑₛₕ by a combination of offsetting and scaling the extremum determined in block 218.

For example, where the block 218 extremum is a minimum Qₘᵢₙ, the block 220 threshold may be determined according to Qₜₕᵣₑₛₕ=Qₘᵢₙ+x, where x is a positive offset value or, the block 220 threshold may be determined according to Qₜₕᵣₑₛₕ=z(Qₘᵢₙ) where z is a positive scaling factor. In other embodiments, where the block 218 extremum is a maximum Qₘₐₓ, the block 220 threshold may be determined according to Qₜₕᵣₑₛₕ=Qₘₐₓ−x where x is a positive offset value or, the block 220 threshold may be determined according to Qₜₕᵣₑₛₕ=z(Qₘₐₓ) where z is a positive scaling factor.

At the conclusion of scan mode 210, controller 170 has determined a threshold level Qₜₕᵣₑₛₕ within the scan region. If the block 218 extremum is a maxi Qₘₐₓ, then the block 220 threshold level Qₜₕᵣₑₛₕ is less than the block 218 maximum Qₘₐₓ. If, on the other hand, the bock 218 extremum is a minimum Qₘᵢₙ, then the block 220 threshold level Qₜₕᵣₑₛₕ is greater than the block 218 minimum Qₘᵢₙ. As explained in more detail below, this threshold level Qₜₕᵣₑₛₕ may be used by controller 170 in indicator mode 212 to determine the location of a vein within the scan region.

In some embodiments, controller 170 may determine a threshold range Qₜₕᵣₑₛₕ_{_{—}}_{range} as a part of block 220, rather than a threshold level Qₜₕᵣₑₛₕ. Such a threshold range Qₜₕᵣₑₛₕ_{_{—}}_{range} may help to avoid spurious results, which may occur for example, because a user directs incident beam into space rather than toward the scan region or because of some other user error. If the block 218 extremum is a maximum Qₘₐₓ, then the block 220 threshold range Qₜₕᵣₑₛₕ_{_{—}}_{range} may be the range Qₜₕᵣₑₛₕ_{_{—}}_{range}≡(Qₘₐₓ−x,Qₘₐₓ+x), where x is a positive offset value or the range Qₜₕᵣₑₛₕ_{_{—}}_{range}≡(z(Qₘₐₓ),y(Qₘₐₓ)), where y, z are positive scaling factors. If the block 218 extremum is a minimum, then the block 220 threshold range Q al may be the range Qₜₕᵣₑₛₕ_{_{—}}_{range}≡(Qₘᵢₙ−x,Qₘᵢₙ+x), where x is a positive offset value or the range Qₜₕᵣₑₛₕ_{_{—}}_{range}≡(z(Qₘᵢₙ),y(Qₘᵢₙ)) where y, z are positive scaling factors.

At the conclusion of scan mode 210, method 200 proceeds to indicator mode 212. A user may effect the change from scan mode 210 to indicator mode 212 using input component 194 (FIG. 1). In block 222, incident beam 12 is again moved across the scan region while receiver 140 detects reflected beam 14. For best results, the scan region used in block 222 is similar to (or the same as) the scan region used in block 216, although this is not necessary. In many respects, block 222 is similar to block 216. However, if a suitable venipuncture site is located in indicator mode 212 prior to completing the block 222 movement of incident beam 12 across the scan region, then it is not necessary to complete the movement of incident beam 12 across the entirety of the scan region.

During block 222, receiver 140 generates receiver output signal 142 in response to detecting reflected beam 14 and passes receiver output signal 142 to controller 170. As discussed above, receiver output signal 142 is representative of one or more characteristics of reflected beam 14. In the illustrated embodiment, method 200 comprises an optional block 224, where controller 170 processes receiver output signal 142 generated in block 222 to detect local extrema of receiver output signal 142 within the scan region. In one particular embodiment, receiver 140 detects the intensity of reflected beam 14 and, as a part of optional block 224, controller 170 processes receiver output signal 142 to detect local minima of the intensity of reflected beam 14 within the scan region.

The local extrema detected in block 224 need not include all of the local extrema in a strictly mathematical sense. For example, controller 170 may be configured to omit local extrema having small variations, such as those which may be caused by noise or the like. Preferably, the local extremum detection procedures of optional block 224 are performed (at lean substantially) in “real time” in relation to the rate at which incident beam 12 is moved across the scan region in block 222. In accordance with such real time operation, when controller 170 detects a local extremum in block 224, incident beam 12 is still in substantially the same location (within the scan region) that created the local extremum.

For each local extremum detected in optional block 224, method 200 proceeds to block 226. Block 226 involves a comparison of a block 224 local extremum to the threshold level Qₜₕᵣₑₛₕ determined in block 220 of scan mode 210. As with block 224, the threshold comparison procedures of block 226 are preferably performed (at least substantially) in “real time” in relation to the rate at which incident beam 12 is moved across the scan region in block 222. In accordance with such real time operation, when controller 170 determines that the threshold level Qₜₕᵣₑₛₕ has been exceeded by the local extremum (block 226 YES output), incident beam 12 is still in substantially the same location (within the scan region) that created the local extremum.

Where the block 220 threshold Qₜₕᵣₑₛₕ is a lower threshold, block 226 involves a determination of whether a block 224 local minimum is less an the block 220 threshold Qₜₕᵣₑₛₕ, If so (block 226 YES output), then controller 170 determines that the current location of incident beam 12 represents the location of a vein and/or a suitable site for venipuncture. Method 200 then proceeds to block 228 where controller 170 causes output component 192 to indicate to the user that the current location of incident beam 12 represents the location of a vein and/or a suitable site for venipuncture. If, on the other hand, a block 224 local minimum is greater than the block 220 threshold level Qₜₕᵣₑₛₕ (block 226 NO output), controller 170 determines that the current location of incident beam 12 is not a location of a suitable vein for venipuncture ad method 200 returns to block 222.

Where the block 220 threshold Qₜₕᵣₑₛₕ is an upper threshold, block 226 involves a determination of whether the block 224 local maximum is greater than the block 220 threshold level Qₜₕᵣₑₛₕ. If so (bock 226 YES output), then controller 170 determines that the current location of incident beam 12 represents the location of a vein and/or a suitable site for venipuncture. Method 200 then proceeds to block 228 where controller 170 causes output component 19 to indicate to the user that the current location of incident beam 12 represents the location of a vein and/or a suitable site for venipuncture. If, on the other hand, a block 224 local min is less than the block 220 threshold level Qₜₕᵣₑₛₕ (block 226 NO output), controller 170 determines that the current location of incident beam 12 is not a location of a suitable vein for venipuncture and method 200 returns to block 222.

In embodiments, where block 220 involves the determination of a threshold range Qₜₕᵣₑₛₕ_{_{—}}_{range} block 226 involves a determination of whether or not the block 224 local maximum falls within the threshold range Qₜₕᵣₑₛₕ_{_{—}}_{range}. If the block 224 local maximum falls within the threshold range Qₜₕᵣₑₛₕ_{_{—}}_{range} (block 226 YES output), then controller 170 determines that the current location of incident beam 12 represents the location of a vein and/or a suitable site for venipuncture. Method 200 then proceeds to block 228 where controller 170 causes output component 192 to indicate to the user that the current location of incident beam 12 represents the location of a vein and/or a suitable site for venipuncture. If, on the other hand, the block 224 local maximum falls outside of the threshold range Qₜₕᵣₑₛₕ_{_{—}}_{range} (block 226 NO output), controller 170 determines that the current location of incident beam 12 is not a location of a suitable vein for venipuncture and method 200 returns to block 222.

Those skilled in the art will appreciate that block 224 is optional. In some embodiments, it is not necessary to determine local extremum in indicator mode 212. In such embodiments, controller 170 directly compares receiver output signal 142 to the block 220 threshold level Qₜₕᵣₑₛₕ (or to the block 220 threshold range Qₜₕᵣₑₛₕ_{_{—}}_{range}) without first having to undergo a local extremum detection process.

As mentioned above, block 228 involves controller 170 causing output component 192 to indicate to the user (e.g. a health care provider) that the current location of incident beam 12 represents the location of a vein or a suitable site for venipuncture. In one particular embodiments, output component 192 comprises a device for marking the epidermis of the patient (e.g. a surgical market or an indentation marker). Accordingly, when controller 170 determines that a particular location within the scan region is a suitable site for venipuncture (block 226 YES output), then this device leaves a mark on the patient's skin such the health care provider can then insert a needle into the patient's vein at the detected venipuncture site. In another embodiment, output component is a LED, some other visible device or an audible device which activates when incident beam 12 is reflected from a suitable site for venipuncture. In this manner, when the visible or audible device is activated, a health came provider can insert a needle at the current location of incident beam 12 to achieve venipuncture.

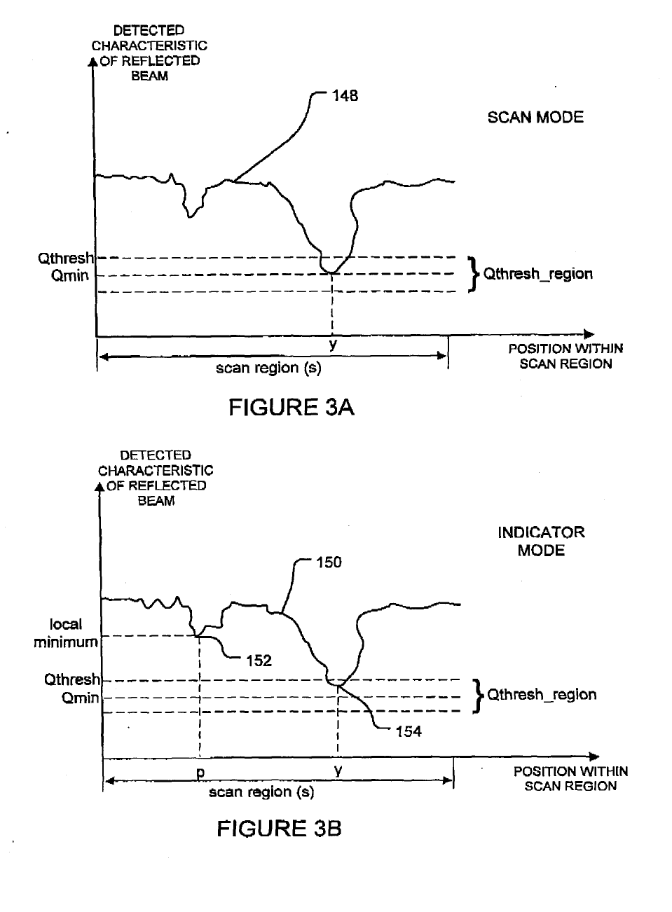

FIGS. 3A and 3B are graphic depictions of data collected in a representative example of method 200 in accordance with a particular embodiment of the invention. FIG. 3A represents data collected in scan mode 210 and FIG. 3B represents data collected in indicator mode 212.

FIG. 3A shows a waveform 148 representing data collected over a scan region s as a part of scan mode 210. The FIG. 3A waveform 148 is a normalized version of receiver output signal 142 generated in block 216 as incident beam 12 is moved across scan region S. In the illustrated example of FIGS. 3A and 3B, the block 218 extremum detection process involves detecting a minimum of waveform 148 and the block 220 threshold setting process involves setting a lower threshold. Waveform 148 has a minimum Qₘᵢₙ at location y with scan region s. Controller 170 ascertains this minimum Qₘᵢₙ as a part of block 218. In block 220, controller 170 then determines a threshold level Qₜₕᵣₑₛₕ in accordance with one of the techniques discussed above. It can be seen in the example of FIG. 3A, that the threshold level Qₜₕᵣₑₛₕ is greater than the block 218 minimum Qₘᵢₙ. At the conclusion of scan mode 210, controller 170 has determined a threshold level Qₜₕᵣₑₛₕ which may be used in indicator mode 212 to determine the location of a vein within scan region s.

As discussed above, in some embodiments, controller 170 determines a threshold range Q a (rather than a threshold level Qₜₕᵣₑₛₕ) in block 220 of scan mode 210. FIG. 3A also shows a suitable threshold range Qₜₕᵣₑₛₕ_{_{—}}_{range}.

FIG. 3B shows a waveform 150 representing data collected over scan region s as a part of indicator mode 212. Scan region s is the same scan region s depicted in FIG. 3A. The FIG. 3B, waveform 150 is a normalized version of receiver output signal 142 generated in block 222 as incident beam 12 is moved across scan region s. It can be seen from FIG. 3B, that waveform 150 has a local minimum 152 at location p with scan region s. As incident beam 12 is moved across scan region s. It can block 222, controller 170 ascertains the local minimum 152 at location p as a part of block 224. Controller 170 then compares the value of local minimum 152 to the block 220 threshold level Qₜₕᵣₑₛₕ as a pan of block 226. Since the value of local minimum 152 is greater than Qₜₕᵣₑₛₕ (block 226 NO output), controller 170 determines that location p is not a suitable site for venipuncture. As discussed above, in some embodiments, block 226 involves a comparison of the value of local minimum 152 to threshold range Qₜₕᵣₑₛₕ_{_{—}}_{range}. Since local minimum 152 falls outside of threshold range Qₜₕᵣₑₛₕ_{_{—}}_{range} (block 226 NO output), controller 170 determines that location y, is not a suitable site for venipuncture. Location p, when compared to location y, may be the site of a small vein or a vein that is deep under the skin of the patient, for example.

Waveform 150 also has another local minimum 154 at location y within scan region s. As incident beam 12 is moved across scan region s in block 222, controller 170 ascertains the local minimum 154 at location y as a part of block 224. Controller 170 then compares the value of local minimum 154 to the block 220 threshold level Qₜₕᵣₑₛₕ as a part of block 226. Since the value of local minimum 154 is less than Qₜₕᵣₑₛₕ (block 226 YES output), controller 170 determines that location y represents the location of a vein and/or a suitable site for venipuncture. In some embodiments, block 226 involves a comparison of the value of local minimum 154 to threshold range Qₜₕᵣₑₛₕ_{_{—}}_{range}. Since local minimum 154 is within threshold range Qₜₕᵣₑₛₕ_{_{—}}_{range} (block 226 YES output), controller 170 determines that location y represents the location of a vein and/or a suitable site for venipuncture.

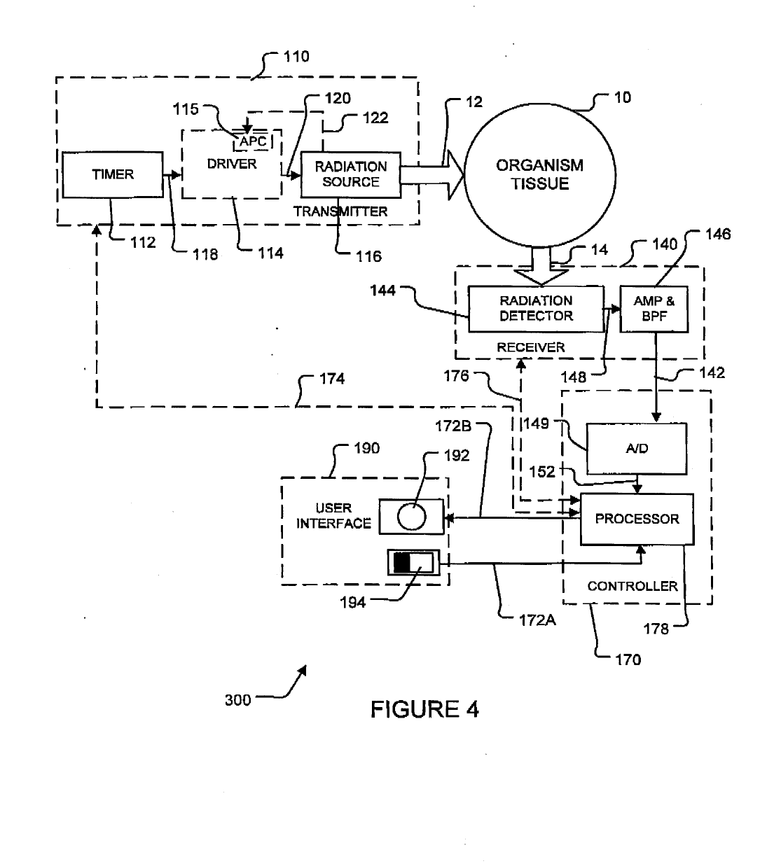

FIG. 4 is a block diagram showing the components of a venipuncture site locator 300 according to another embodiment of the invention. In many respects, site locator 300 (FIG. 4) is similar to site locator 100 (FIG. 1) and similar reference numerals are used to refer to similar features of site locator 300 and site locator 100.

In site locator 300 of FIG. 4, transmitter 110 comprises a timer 112, a driver 114 and a radiation source 116. When activated (by controller signal 174 for example), timer 112 generates a pulse train sib 118. Driver 114 receives pulse train signal 118 and, in response to pulse train signal 118, generates a driver signal 120 which is provided to radiation source 116. In response to receiving drive signal 120, radiation source 116 outputs incident radiation beam 12.

Timer 112 may be adjustable (by controller signal 174 for example) to vary the amplitude, frequency and/or duty cycle of pulse train signal 118. In some embodiments, pulse train signal 118 has a frequency in a range of 1 kHz-1 MHz. In some embodiment, pulse train signal 118 has a frequency in a range of 1-50 kHz. The frequency of pulse train signal 118 may be selected on the basis of one or more of: the response time of receiver 140, the frequency of ambient noise in the application environment; the frequency of ambient light which may cause interference; and the ability to sample received signal 14 at the frequency of pulse train signal 118. Preferably, the frequency of pulse train signal 118 is maintained substantially constant over a particular vein location operation.

Driver 114 receives pulse train signal 118 and provides driver signal 120. Driver 114 may comprise one or more suitably configured amplification stages which may help to source current for driving radiation source 116. Driver signal 120 may exhibit the frequency and/or duty cycle of pulse train signal 118.

Radiation source 116 receives driver signal 120 and, in response to driver signal 120, outputs incident radiation beam 12. In site locator 300, radiation source 116 causes incident radiation beam 12 to be modulated by the frequency of pulse train signal 118 and driver signal 120. In currently preferred embodiments, incident radiation beam 12 emitted by radiation source 116 is in the infrared or near infrared spectrum. For example, incident radiation beam 12 may have a frequency in a range of 600-1,000 mm. As discussed above, at this frequency, the reflectance of blood may be significantly different than other soft tissue or hard tissue that may be located in organism tissue 10. In some embodiments, radiation source 116 comprises a light emitting diode (LED). In other embodiments, radiation source 116 comprises a laser diode. Those skilled in the art will appreciate that there are a wide variety of commercially available laser diodes capable of providing the functionality of radiation source 116.

In some embodiments, driver 114 comprises an optional automatic power control (APC) circuit 115 which incorporates feedback 122 from radiation source 116 and uses feedback 122 to controllably adjust driver signal 120 and to thereby controllably maintain a desired power level for incident radiation beam 12. APC circuit 115 is useful to maintain a constant power for incident beam 12, even where emission of incident beam 12 causes radiation source 116 to heat up. APC circuit 115 may comprise a radiation detector (not shown), such as a photodiode or a phototransistor, to provide feedback 122. Feedback 122 is preferably correlated with the output power of incident radiation beam 12 emitted from radiation source 116. APC circuit 115 may also be adjustable, so as to vary the desired output power of incident radiation beam 12 emitted from radiation source 116. In some embodiments, APC circuit 115 may be manually adjusted to vary die output power level of radiation source 116 (e.g. using an input component 194 on user interface 190). In other embodiments, controller 170 may adjust APC circuit 115 to vary the output power level of radiation source 116. Those skilled in the art will appreciate that there are a wide variety of commercially available APC circuits which are sold in conjunction with laser diodes and which are capable of providing the functionality of APC circuit 115.

Radiation source 116 may comprise or otherwise be provided with suitable optics (not shown in FIG. 4) to control the characteristics of incident radiation beam 12, such as the divergence, collimation and/or spot size of incident radiation beam 12, for example. It may be desirable to use optical techniques to adjust the characteristics of incident radiation beam 12 such that it does not cause significant damage to organism tissue 10. For example, it may be desirable to optically increase the spot size of incident radiation beam 12 when it interacts with organism tissue 10, such that the power density of incident radiation beam 12 is sufficiently low to avoid significant damage to organism tissue 10. APC circuit 115 can also be used to control the out power of incident radiation beam 12. Preferably, the power density of incident radiation is less than about 5 W/cm².

In some embodiments, transmitter 110 may also comprise a cooling system (not shown) in FIG. 4 to cool radiation source 116 and/or driver 114. Such a cooling system may comprise one or more heat sinks and/or means for creating a flow of air in the vicinity of transmitter 110.

Incident radiation beam 12 generated by transmitter 110 impinges on organism tissue 10 and reflects from the structures in organism tissue 10 to produce reflected radiation beam 14. Reflected radiation beam 14 is received by receiver 140.

In site locator 300 of FIG. 4, receiver 140 comprises: a radiation detector 144 which receives reflected radiation beam 14 and outputs a detector signal 148; and signal conditioning circuitry 146, which conditions detector signal 148 to generate receiver output signal 142. Radiation detector 144 may generally comprise any suitably configured radiation sensor capable of receiving electromagnetic radiation and generating an electrical signal responsive to the received electromagnetic radiation. Preferably, radiation detector 144 is sensitive to radiation at the wavelength of reflected radiation beam 14. In addition, the response time of radiation detector 144 should be sufficiently fast to accommodate the modulation frequency of radiation beam 12, 14 (i.e. the frequency of pulse train signal 118). In some embodiments, radiation detector 144 comprises a suitably configured photodiode. In other embodiments, radiation detector 144 comprises suitably configured phototransistor. Those skilled in the art will appreciate that there are a wide variety of commercially available phototransistors capable of providing the functionality of radiation detector 144.

The output of radiation detector 144 (detector signal 148) is received at signal conditioning circuitry 146. In the illustrated embodiment of site locator 300, signal conditioning circuitry 146 amplifies and filters detector signal 148 in the analog domain to produce an analog receiver output signal 142. Preferably, the filtration operations performed by signal conditioning circuitry 146 comprise band pass filtering with a center frequency that is substantially similar to the frequency of pulse train signal 118 and a relatively sharp out-of-band rejection slope. In some embodiments, the pass band of the filtration operations (as defined by its corner frequencies) is less than 10 kHz. In some embodiments, the out-of-band rejection slope of the filtration operations is greater than 40 dB/dec. Preferably, the amplification operations performed by signal conditioning circuitry 146 have a gain on the order of 10² or greater. As discussed above, signal conditioning circuitry 146 may also comprise one or more analog to digital converters, such that detector signal 148 (or an amplified version of detector signal 148) is sampled and digitized prior to becoming receiver output signal 142. In some embodiments, various signal conditioning operations (such as filtering, for example) may be performed in the digital domain.

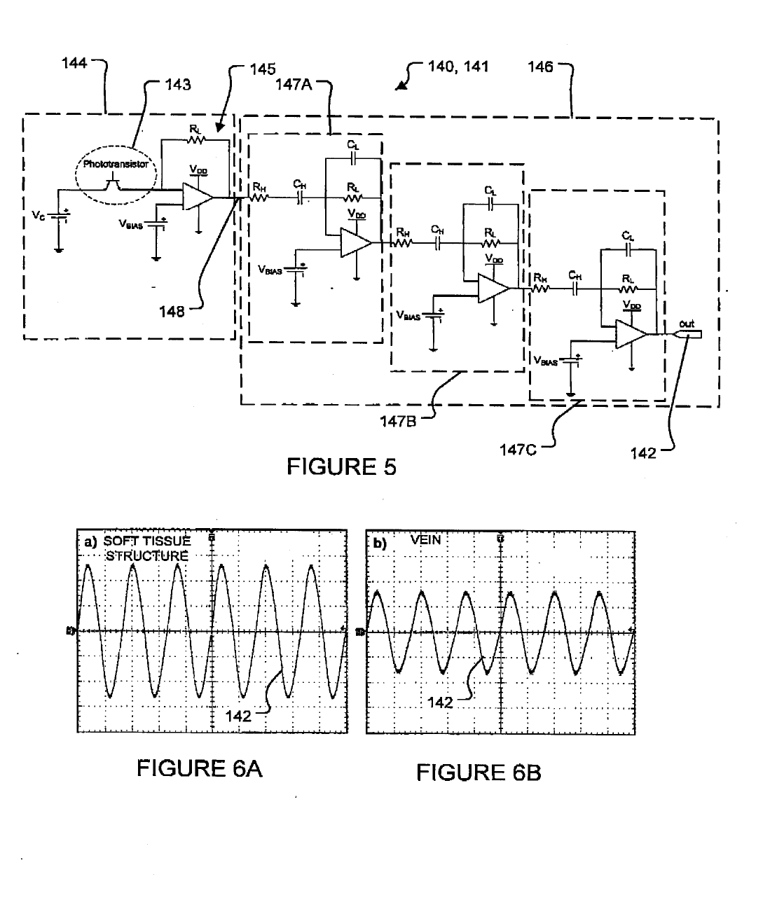

FIG. 5 shows a detailed example of a circuit 141 suitable for implementing receiver 140 of FIG. 4. In circuit 141 of FIG. 5, radiation detector 144 comprises a phototransistor 143 and current-to-voltage conversion circuitry 145. In some embodiments, radiation detector 144 may be implemented using one or more radiation detectors of other types, such as a photodiode, for example. When reflected radiation beam 14 is incident on phototransistor 143, phototransistor 143 conducts current. This current is converted to a voltage signal (detector signal 148) by current-to-voltage conversion circuitry 145. In circuit 141 of FIG. 5, signal condition circuitry 146 comprises a plurality of amplification and band pass filtration stages 147A, 147B, 147C. In some embodiments, each amplification and band pass nitration stage 147A, 147B, 147C is substantially identical. Together, band pass filtration stages 147A, 147B, 147C form a third order baud pass filter which may be configured by adjusting the values of resistors R_{L}, R_{H} and capacitors C_{L}, C_{H}. In one particular embodiment, where the modulation frequency of radiation beams 12, 14 is 30 kHz, band pass filtration stages 147A, 147B, 147C are configured to provide a total gain of 10³, corner frequencies of 27 kHz and 33 kHz and an out-of-band rejection slope of 60 dB/dec. In alternative embodiments, signal conditioning circuitry 146 may have a different number of amplification and filtration stages.

The output of signal conditioning circuitry 146 is receiver output signal 142, which is provided to controller 170. FIGS. 6A and 6B depict exemplary receiver output signals 142. In FIG. 6A, incident beam 12 is directed toward a soft tissue structure of organism tissue 10, and in FIG. 6B, incident beam 12 is directed toward a vein within organism tissue 10. FIGS. 6A and 6B show that receiver output signal 142 exhibits the same frequency (i.e. the modulation frequency of pulse train signal 118) whether incident beam is directed at a soft tissue structure (FIG. 6A) or at a vein (FIG. 6B). Comparing FIGS. 6A and 6G, it can also be seen that receiver output signal 142 of FIG. 6A exhibits a relatively high amplitude and receiver output signal 142 of FIG. 6B exhibits a relatively low amplitude. As discussed above, this amplitude difference between receiver output signals in FIG. 6A and FIG. 6B occurs because, at the wavelength of infrared or near-infrared radiation, the reflectance of soft tissue structures (FIG. 6A) is significantly higher than the reflectance of blood and veins (FIG. 6B). Table 1 shows typical reflectance levels of various structures within organism tissue 10 (to infrared or near infrared radiation).

TABLE 1
Interaction of Tissue Structures with Infrared Radiation

| Structure | Transmission | Absorption | Reflectance |
|---|---|---|---|
| Soft Tissue | 10% | 25% | 65% |
| Hard Tissue | 10% | 10% | 80% |
| Blood | 25% | 35% | 40% |

In site locator 300 of FIG. 4, controller 170 comprises: an A/D converter 149 which samples and digitizes receiver output signal 142 to provide digital received signal 152; and processor 178 which controls the operation of site locator 300. In the illustrated embodiment, processor 173 is an embedded microprocessor, but those skilled in the art will appreciate that processor 178 may be implemented using a wide variety of suitably configured processing devices. Processor 178 may optionally control the operation of transmitter 110 using control signal 174 and may optionally control the operation of receiver 140 using control signal 176. In the illustrated embodiment, optional control signals 174, 176 are shown as two-way control signals, but this is not necessary. Control signals 174, 176 may have functions similar to those discussed above for site locator 100 of FIG. 1.

Processor 178 may also interact with user interface 190 using control signals 172A, 172B. User interface 190 is substantially similar to user interface 190 of FIG. 1 site locator 100. User interface 190 comprises an input component 194 which may communicate with processor 178 via control signal 172A. A user may use input component 194 to turn processor on and off, to switch site locator 300 between scan mode and indicator mode or to otherwise control the operation of processor 178. User interface 190 also comprises an output component 192. Output component 192 indicates to users when site locator 300 has located a vein which may be suitable for venipuncture. As with site locator 100 of FIG. 1, output component 192 may comprise a ski marker, a LED, some other type of visible device, an audible device or some other sort of indicator which indicates the presence of a suitable venipuncture site. Processor 178 controls the operation of output component 192 via control signal 172B.

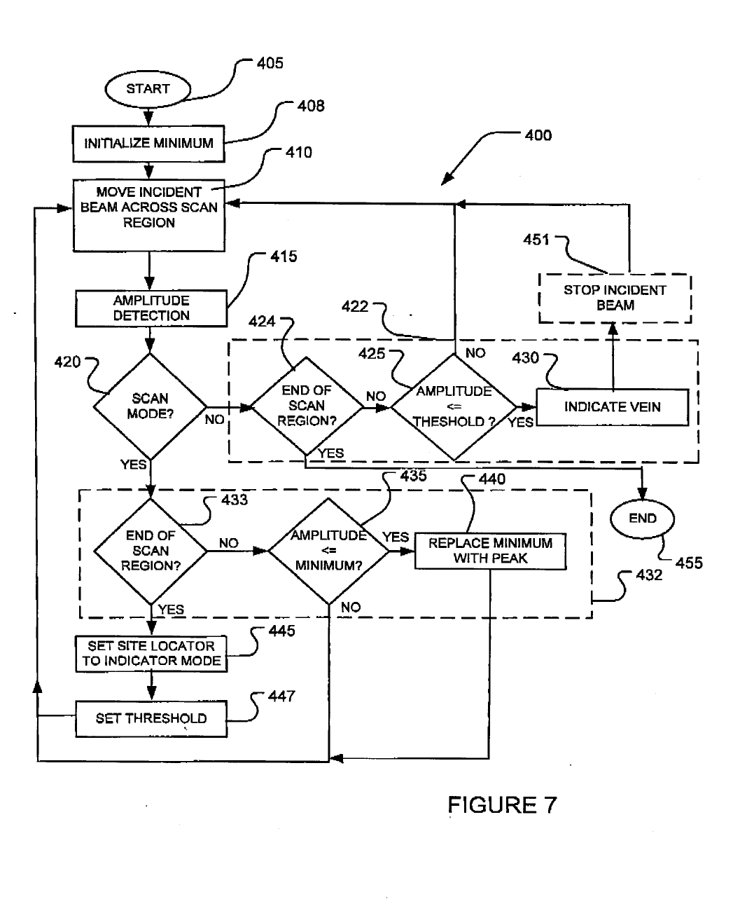

FIG. 7 schematically depicts a method 400 of using site locator 300 (FIG. 4) to locate vein(s) and suitable venipuncture site(s) in accordance with another embodiment of the invention. In many respects, method 400 of FIG. 7 is similar to method 200 of FIG. 2. Method 400 starts in block 405, where site locator 300 is set to scan mode. As discussed above, a user may configure site locator 300 to be in scan mode using input component 194. After entering scan Mode in block 405, method 400 proceeds to block 408 where processor 178 initializes a minimum value variable (MIN). Block 408 may involve initializing the minimum value (Mm) to have the maximum possible value of digital received signal 152. In some embodiments, the block 408 initialization value represents the highest output value of A/D converter 149. For example, where A/D converter 149 is an n bit A/D converter, the block 408 initialization value is 2ⁿ−1 (i.e. if A/D converter 149 is an 8 bit A/D converter, then the block 408 initialization value is 2⁸−1=255).

After initializing the minimum value (MIN) in block 408, method 400 proceeds to block 410, where incident beam 12 is moved across a scan region. In some embodiments, a user moves site locator 300 across the scan region (or site locator 300 is otherwise caused to move across the scan region) and incident beam 12 moves with site locator 300. In other embodiments, incident beam 12 moves independently of site locator 300 (e.g. incident beam 12 moves in response to a moveable radiation source 116 or moveable optical elements (not shown)). The upper limit on the rate of movement of incident beam 12 in block 410 may be determined by the user's ability to react to output component 192 indicating the presence of a vein. For example, if output component 192 is a LED which indicates the presence of a vein at a particular location, then the rate of movement of incident beam 12 across the scan region during indicator mode should be sufficiently slow so that a user can determine the location indicated by activation of the LED. In some embodiments, the rate of movement of incident beam 12 across the scan region in indicator mode is less than 10 cm/sec. In some embodiments, this rate is less than 5 cm/sec. While the rate of movement of incident beam 12 across the scan region in scanning mode is not constrained by the user reaction time, the rate of movement of incident beam 12 in scanning mode may also be less than 10 cm/sec.

While incident beam 12 is being moved across the scan region in block 410, receiver 140 detects reflected beam 14 and generates receiver output signal 142. Block 415 involves detecting the amplitude of receiver output signal 142 in real time. FIGS. 6A and 68 depict exemplary receiver output signals 142 which are modulated by the frequency of pulse train signal 118. As discussed above, the amplitude of receiver output signal 142 is correlated to the reflectance of the structures in organism tissue 10. In some embodiments, the block 415 amplitude detection process involves sampling and digitizing receiver output signal 142 and then detecting its peak. The sampling and digitizing of receiver output signal may be implemented by A/D converter 149 and detecting the peak of the resultant digital received signal 152 may be implemented by processor 178.

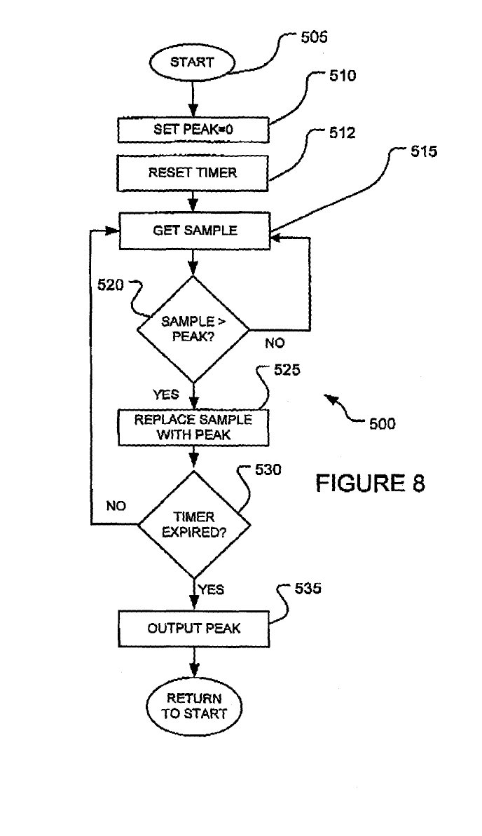

FIG. 8 depicts an exemplary method 500 of implementing the block 415 amplitude detection process by sampling, digitizing and detecting the peak of receiver output signal 142 in accordance with a particular embodiment of the invention. Method 500 begins in block 505 and then proceeds to block 510, where a PEAK value variable is initialized to zero. After initializing the PEAK value, method 500 proceeds to block 512, where a peak detect timer is reset. Block 515 involves obtaining and digitizing a sample of receiver output signal 142. In site locator 300 of FIG. 4, A/D converter 149 implements the sampling and digitizing of block 515. To effect this sampling and digitizing, A/D converter 149 may be controlled by processor 178 or some other timing component (not shown). Once a digital sample is obtained in block 515, processor 178 compares the current sample to the PEAK value in block 520. If the current sample is less than or equal to the PEAK value (block 520 NO out), then method 500 returns to block 515 to obtain another sample.

If, on the other and, the current sample is greater than the PEAK value (block 520 YES output), then method 500 proceeds to block 525. In block 525, processor 178 replaces the PEAK value with the Current sample. Method 500 then proceeds to block 530, where controller 178 determines whether the pa detect timer has expired. If the peak detect timer has not expired (block 530 NO output), then method 500 loops back to block 515 to obtain another sample. If, on the other hand, the peak detect timer has expired (block 530 YES output), then method 500 proceeds to block 535. In block 535, the PEAK value is output as the amplitude of the block 415 amplitude detection process. Method 500 then returns to block 505.

The sampling rate of method 500 (i.e. the length of time required to loop through blocks 515-530) may depend on the modulation rate of receiver output signal 142, which in the depends on the frequency of pulse train signal 118. Preferably, the ratio of the method 500 sampling rate to the modulation frequency of receiver output signal 142 is 10:1 or greater. In some embodiments, this ratio is 100:1 or greater, In some circumstances, an increase in this ratio will result in method 500 providing improved accuracy to the block 415 amplitude detection process. However, in practice the upper bound of the method 500 sampling rate is limited by the cost and availability of suitable sampling and digitizing hardware and the lower bound of the modulation frequency is limited by noise considerations. In some embodiments, where the period of the peak detect timer is longer and the sampling frequency is asynchronous with the modulation frequency, it is possible to achieve sufficiently accurate amplitude detection where the ratio of the method 500 sampling rate to the modulation frequency of receiver output signal 142 is less than 10:1.

The period of the peak detect timer used in method 500 way depend on the modulation frequency of receiver output signal 142, the rate at which incident beam 12 is moved across the scan region (block 410) and the required accuracy of site locator 300. Those skilled in art will appreciate that if the modulation frequency is relatively low, then the period of the method 500 peak detect timer is preferably relatively high, so that the method 500 peak detect period lasts at least one full modulation period of receiver output signal 142. This constraint on the peak detect timer period ensures that at least one peak of receiver output signal 142 occurs within the method 500 peak detect timer period. For a given site location accuracy requirement, if incident beam 12 is moved quickly across the scan region (in block 410), then the period of the method 500 peak detect timer is preferably relatively low. Conversely, if the incident beam 12 is moved relatively slowly across the scan region (in block 410), then the method 500 peak detect timer period may be increased. This constraint on the peak detect timer period ensures that method 500 detects the amplitude of receiver output signal 142 in “real time” (i.e. before incident beam 12 has moved too far from its current location). In some embodiments, the peak detect timer period is in a range of 20 μs-20 ms.

Those skilled in the art will appreciate that method 500 of FIG. 8 represents only one of many possible amplitude detection techniques which may be used to implement the block 415 amplitude detection process (FIG. 7). Once receiver output signal 142 is sampled and digitized to provide digital received signal 152, processor 178 may implement other algorithms to detect the amplitude of digital received signal 152. In other embodiments, the block 415 peak detection process may be implemented in the analog domain using suitable analog circuitry, such as an envelope detector circuit, a peak rectifier circuit or the like. In still other embodiments, receiver output signal 142 may be demodulated in the analog domain (i.e. to remove the modulation introduced by pulse train signal 118). Demodulating receiver output signal 142 in the analog domain will yield a demodulated signal whose amplitude varies with the reflectance of the structures in organism tissue 10. In such embodiments, the block 415 amplitude detection process may be implemented by sampling the demodulated signal without requiring a peak detect process.

After detecting the amplitude of receiver output signal 142 in block 415, method 400 (FIG. 7) proceeds to block 420, where processor 178 determines whether site locator 300 is in scan mode or indicator mode. If site locator 300 is in scan mode (block 420 YES output), then method 400 proceeds to scan mode loop 432. If, on the other hand, site locator 300 is in indicator mode (block 420 NO output), then method 400 proceeds to indicator mode loop 422. Assuming, that incident beam 12 is being moved across the scan region (block 410) for the first time, site locator 300 will be in scan mode and so method 400 will proceed to block 433 of scan mode loop 432.

Block 433 involves querying whether incident beam 12 has reached the end of the scan region or if site locator 300 has otherwise been switched from scan mode into indicator mode. If incident beam 12 has not reached the end of the scan region and site locator 300 has not otherwise been set to indicator mode (block 433 NO output), then method 400 proceeds to block 435.

Block 435 involves comparing the current block 415 amplitude value to the minimum value (MIN). On the first the through scan mode loop 432, the minimum value (MIN) has die value with which it was initialized in block 408 (i.e. the highest possible value of digital received signal 152). Consequently, on the first nine through scan mode loop 432, the current block 415 amplitude value will always be less than or equal to the minimum value (MIN) and method 400 will always exit block 435 via the block 435 YES output. On subsequent iterations of scan mode loop 432, the current block 415 amplitude value may be greater than, less than or equal to the minimum value (MIN). If the block 435 comparison indicates that the current block 415 amplitude value is greater than the minimum value (MIN), then method 400 loops back to block 410 (block 435 NO output) and incident beam 12 continues to move across the scan region.

If, on the other hand, the block 435 comparison indicates that the current block 415 amplitude is less than or equal to the mum value (MIN), then method 400 proceeds to block 440 (block 435 YES output). In block 440, processor 178 replaces the minimum value (with the current block 415 amplitude value. In this manner, scan mode loop 432 operates to update the minimum value (MIN) to reflect the lowest value of the block 415 amplitude that has been detected while site locator 300 is in its scan mode. After block 440, method 400 loops back to block 410 and incident beam 12 continues to move across the scan region.

Returning to block 433, if the result of the block 433 inquiry determines that incident beam 12 has reached the end of the scan region or if site locator 300 has otherwise been switched from scan mode into indicator mode (block 433 YES output), then method 400 proceeds to block 445. In some embodiments, the block 433 inquiry results in a YES output because a user has set site locator 300 to indicator mode (via input component 194 for example). In block 445, processor 178 sets site locator 300 to indicator mode (if site locator 300 has not already been set to indicator mode by a user). From block 445, method 400 proceeds to block 447, where processor 178 sets the threshold level Qₜₕᵣₑₛₕ which will be used in the subsequent indicator mode. The block 447 threshold level Qₜₕᵣₑₛₕ is preferably based on the minimum value (MIN) determined in scan mode loop 432. In some embodiments, the threshold level Qₜₕᵣₑₛₕ is determined by offsetting and/or scaling the minimum value (MIN). For example, the threshold level Qₜₕᵣₑₛₕ may be given by Qₜₕᵣₑₛₕ=MIN+x, where x is a positive offset value or the threshold level Qₜₕᵣₑₛₕ may be given by Qₜₕᵣₑₛₕ=z(MIN), where z is a positive scaling factor.

As discussed above, in some embodiments, it is preferably to have a threshold range Qₜₕᵣₑₛₕ_{_{—}}_{range} rather than a threshold level, to avoid spurious results. In some embodiments, the threshold range represents a range surrounding the value (MIN) which may be determine by offset and/or scaling the minimum value (MIN). In some embodiments, the block 447 threshold range Qₜₕᵣₑₛₕ_{_{—}}_{range} is given by Qₜₕᵣₑₛₕ_{_{—}}_{range}≡(MIN−x, MIN+x), where x is a positive offset value or the range Qₜₕᵣₑₛₕ_{_{—}}_{range}≡(z(MIN), y(MIN)) where y, z is a positive scaling factors.

After block 447, method 400 loops back to block 410, where incident beam 12 is again moved across the scan region, but this time site locator 300 is in indicator mode rather than scan mode. Accordingly, when incident beam is moved across the scan region a second time (block 410), method 400 detects the current amplitude of receiver output signal 142 in block 415 and then proceeds to block 420. The block 420 inquiry determines that site locator 300 is in its indicator mode and, consequently, method 400 proceeds to block 424 of indicator mode loop 422 (block 420 NO output).

Block 424 involves querying whether incident beam 12 has reached the end of the scan region or if site locator 300 has otherwise been switched off or out of indicator mode. If incident beam 12 has reached the end of the scan region or site locator 300 has otherwise been switched off or out of indicator mode (block 424 YES output), then method 400 ends in block 455. If, on the other hand, incident beam 12 has not reached the end of the scan region and site locator 300 has not otherwise been switched off or out of indicator mode (block 424 NO output), then method 400 proceeds to block 425.

In block 425, processor 178 compares the current block 415 amplitude with the block 447 threshold Qₜₕᵣₑₛₕ determined in during the scan mode. If the current block 415 amplitude is greater than the block 447 threshold Qₜₕᵣₑₛₕ (block 425 NO output), then method 400 loops back to block 410 where incident beam 12 continues to move across the scan region. If, on the other hand, the current block 415 amplitude is less than or equal to the block 447 threshold Qₜₕᵣₑₛₕ (block 425 YES output), then method 400 proceeds to block 430 where processor 178 causes site locator 300 to indicate the presence of a vein. As discussed above, site locator 300 may comprises a variety of different types of output components 192, such as an LED, another type of visible output, a skin marker (e.g. a surgical marker or an indentation marker) or an audible output, which may be used to indicate the presence of a vein in block 430 and processor 178 may activate output component 192 using signal 1723

In some embodiments, where block 447 determines a threshold range Qₜₕᵣₑₛₕ_{_{—}}_{range} block 425 involves an inquiry into whether the Current block 415 amplitude falls within the threshold range Qₜₕᵣₑₛₕ_{_{—}}_{range}. If the current block 415 amplitude the outside of the threshold range Qₜₕᵣₑₛₕ_{_{—}}_{range}, then method 400 loops back to block 410 (block 425 NO output), where incident beam 12 continues to move across the scan region. If, on the other hand, the current block 415 amplitude falls within the threshold range Qₜₕᵣₑₛₕ_{_{—}}_{range} then method 400 proceeds to block 430 (block 425 YES output), where processor 178 cause site locator 300 to indicate the presence of a vein.

When site locator 300 indicates the presence of a vein in block 430, incident beam 12 may optionally be stopped from across the scan region in block 451 (i.e. such that incident beam 12 stays in the indicated vein location). In this manner, incident beam 12 does not move from the detected venipuncture site. In circumstances where incident beam 12 is moving autonomously, processor 178 may cause incident beam 12 to stop moving in block 451. In other embodiments, where a user is causing incident beam 12 to move across the scan region, the user may cause incident beam 12 to stop moving in block 451. Stopping the movement of incident beam 12 in block 451 may be useful where output component 192 is a LED or an audible device, for example, because output component 192 can remain active at the detected venipuncture site until venipuncture has been achieved or the venipuncture site has been marked (e.g. with a surgical marker). In other embodiments, where output component 192 comprises an epidermal marker, for example, it is not necessary to stop incident beam 12, as the epidermal marker will leave a mark where site locator 300 has detected a vein.

In some embodiments, method 400 may end after a vein has been indicated in block 430 (or after incident beam 12 has been stopped (block 451)). However, in other embodiments, after block 430 or optional block 451, method 400 loops back to block 410, where incident beam 12 continues to move across the scan region. Where method 400 loops back to block 410 in this manner, it may be possible to detect the presence of multiple veins and/or venipuncture sites in a scan region.

In the illustrated embodiment, the procedures of many of the blocks in method 400 are performed in “real time”, while site locator 300 is being moved across the scan region in block 410. The accuracy with which a user will be able to predict the location of a vein using site locator 300 will generally depend on the speed with which incident beam 12 is moved across the scan region in block 410, the speed of the real time processing in method 400 and the speed and precision with which a user (or controller 178) reacts to the block 430 indication of a vein. To achieve a real time effect, the processing involved in each loop through method 400 is preferably fast in relation to the block 410 rate of movement of incident beam 12 across the scan region. Preferably, a loop through method 400 is sufficiently fast that it will accurately detect the location of a vein to within 0.01 cm. Accordingly, if the rate of movement of incident beam 12 in block 410 during indicator mode is 10 cm/sec, then the time for a loop through the procedures of blocks 415-430 is less than 0.001 seconds. Similarly, where the movement of incident beam 12 in block 410 in indicator mode is implemented by a user, a user will typically take at least 0.1 seconds to react to the block 430 vein indication. Preferably the time for a loop through the procedures of blocks 415-430 is at least an order of magnitude greater than this user reaction time.

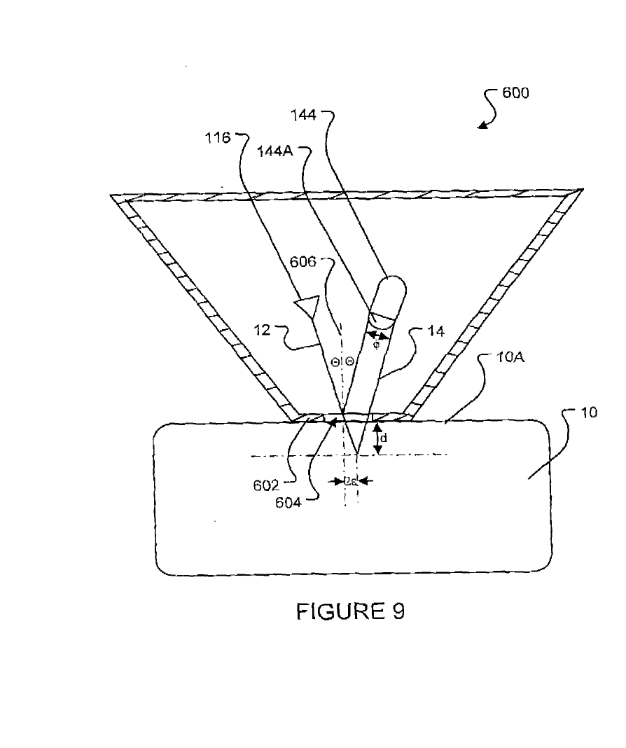

FIG. 9 schematically depicts a portion of a housing 600 for site locator 300 according to a particular embodiment of the invention Housing 600 encloses radiation source 116 and radiation detector 144. Housing 600 may enclose other components of site locator 300 which are not shown in FIG. 9. In the illustrated embodiment of FIG. 9, housing 600 comprises a face 602 which contacts the epidermis 10A of organism tissue 10 in the scan region. Incident radiation beam 12 emitted by radiation source 116 passes through face 602 and impinges on organism tissue 10. Reflected beam 14 is reflected from organism tissue 10, through face 602 and to radiation detector 144. In some embodiments, face 602 may define an aperture 604 through which incident radiation beam 12 and reflected radiation beam 14 may travel. Aperture 604 may comprise a lens (not shown). In the illustrated embodiment of FIG. 9, incident beam 12 is moved across the scan region by moving housing 600.

In the illustrated embodiment, radiation detector 144 comprises a lens 144A having a lens diameter (aperture) φ. In the illustrated embodiment, radiation source 116 is mounted such that incident beam 12 forms an angle of incidence Θ with respect to the normal 606. Analyzing the geometry of FIG. 9, it can be see that if it is desired to locate veins at a depth d below the epidermis 10A of organism tissue 10 with a margin of error less than or equal to ε, the angle of incidence Θ is given by:

$$Θ = \frac{1}{2} {sin}^{- 1} ( \frac{φ}{\sqrt{{d}^{2} - 4 {ɛ}^{2}}} )$$

It is desirable in some applications to limit the penetration distance d, so that site locator 300 does not detect arteries which are typically located deeper within organism tissue 10. In some embodiments, the penetration distance d is in a range of 0-10 mm. In other embodiments, the penetration distance d is in a range of 0-15 min. However, the desired penetration distance d will typically depend on the type of organism and the location of the scan region on a particular organism for which site locator 300 is employed. The penetration distance d is not limited to these ranges.

While a number of exemplary aspects and embodiments have been discussed above, those of skill in the art will recognize certain modifications, permutations, additions and sub-combinations thereof. For example:
In some embodiments, it may be desirable to reduce the spot size of incident beam 12 on organism tissue 10. In such embodiments, site locator 300 may comprise a Kepler telescope configuration in the optical path of incident beam 12. A Kepler telescope configuration comprises a pair of convex lenses separated by a distance d that is the sum of the focal length of the two lenses. The convex lenses comprise an input lens and an output lens and may be plano-convex or bi-convex lenses. The ratio of the focal length of the output lens to the focal length of the input lens will cause a corresponding ratio in spot size reduction.
In some embodiments, it may be possible to use a transmitted energy beam rather than a reflected energy beam. The invention should be understood to incorporate embodiments based on variation in the transmittance of energy beams between the various types of structures in organism tissue 10.
   Accordingly, the scope of the invention should be construed in accordance with the substance defined by the following claims.

1. A method for locating a vein within organism tissue, the method comprising:
directing an incident energy beam toward the organism tissue, at least a portion of the incident energy beam reflecting from structures within the organism tissue as a reflected energy beam;
moving the incident energy beam across a scan region of the organism tissue a first time and detecting a corresponding first reflected energy beam, thereby generating a first signal representative of at least one characteristic of the first reflected energy beam;
identifying an extremum of the first signal and determining a threshold level based on the identified extremum;
moving the incident energy beam across the scan region a second time and detecting a corresponding second reflected energy beam, thereby generating a second signal representative of at least one characteristic of the second reflected energy beam; and
while moving the incident energy beam across the scan region the second time, locating a vein on the basis of comparing the second signal to the threshold level.

2. A method according to claim 1 wherein directing the incident energy beam toward the organism tissue comprises directing an incident electromagnetic radiation beam having a wavelength in a range of 600-1,000 nm toward the organism tissue.

3. (canceled)

4. A method according to claim 2 comprising modulating the incident electromagnetic radiation beam with a carrier frequency in a range of 1-50 kHz.

5. (canceled)

6. A method according to claim 1 wherein moving the incident energy beam across the scan region the first time and moving the incident energy beam across the scan region the second time comprise autonomously moving the incident energy beam across the scan region.

7. (canceled)

8. A method according to claim 1 wherein moving the incident energy beam across the scan region the first time and moving the incident energy beam across the scan region the second time are performed by a user.

9. A method according to claim 1 wherein moving the incident energy beam across the scan region the first time and moving the incident energy beam across the scan region the second time comprise moving the incident energy beam across the scan region at a rate less than 10 cm/second.

10.-16. (canceled)

17. A method according to claim 1 wherein identifying the extremum of the first signal comprises identifying a minimum of the first signal and wherein identifying the minimum of the first signal comprises:
(a) determining a current amplitude value of the first signal;
(b) comparing the current amplitude value to a minimum value variable (MIN);
(c) if the current amplitude value is less than the minimum value variable (MIN), replacing the minimum value variable (MIN) with the current amplitude value;
(d) repeating steps (a), (b) and (c) while moving the incident energy beam across the scan region the first time; and
(e) after moving the incident energy beam across the scan region the first time, assigning the minimum value variable (MIN) to be the minimum of the first signal.

18. A method according to claim 17 wherein a time for a single repetition of steps (a), (b) and (c) is less than a time required to move the incident energy beam across the scan region by a distance of 0.01 cm.

19. (canceled)

20. (canceled)

21. A method according to claim 4 wherein identifying the extremum of the first signal comprises identifying a minimum of the first signal and wherein identifying the minimum of the first signal comprises:
(a) sampling and digitizing the first signal for a peak detect timer period to obtain a first digital signal;
(b) obtaining a maximum value of the first digital signal;
(c) comparing the maximum value of the first digital signal to a minimum value variable (MIN);
(d) if the maximum value of the first digital signal is less than the minimum value variable (MIN), replacing the minimum value variable (MIN) with the maximum value of the first digital signal;
(e) repeating steps (a), (b), (c) and (d) while moving the incident energy beam across the scan region the first time; and
(f) after moving the incident energy beam across the scan region the first time, assigning the minimum value variable (MIN) to be the minimum of the first signal.

22. A method according to claim 21 wherein sampling and digitizing the first signal is performed at a sampling rate that is 10 or more times the carrier frequency.

23. (canceled)

24. A method according to claim 21 wherein the peak detect timer period is greater than a period of the carrier frequency.

25.-27. (canceled)

28. A method according to claim 1 wherein determining the threshold level based on the identified extremum comprises one or more of: offsetting the identified extremum to arrive at the threshold level; and scaling the identified extremum to arrive at the threshold level.

29. (canceled)

30. (canceled)

31. A method according to claim 1 wherein comparing the second signal to the threshold level comprises:
(a) determining a current amplitude value of the second signal;
(b) determining whether the current amplitude value of the second signal is less than the threshold level; and
(c) repeating steps (a) and (b) while moving the incident energy beam across the scan region the second time.

32. A method according to claim 31 wherein a time for a single repetition of steps (a) and (b) is less than a time required to move the incident energy beam across the scan region by a distance of 0.01 cm.

33. (canceled)

34. (canceled)

35. A method according to claim 31 wherein locating the vein comprises indicating that a current location of the incident energy beam represents a location of the vein, if the current amplitude value of the second signal is determined to be less than the threshold level.

36. A method according to claim 1 wherein comparing the second signal to the threshold level comprises:
(a) sampling and digitizing the second signal for a peak detect timer period to obtain a second digital signal;
(b) obtaining a maximum value of the second digital signal;
(c) determining whether the maximum value of the second digital signal is less than the threshold level; and
(d) repeating steps (a), (b) and (c) while moving the incident energy beam across the scan region the second time.

37.-39. (canceled)

40. A method according to claim 1 wherein determining the threshold level based on the identified extremum comprises determining a threshold range, the threshold range extending above and below the identified extremum and wherein comparing the second signal to the threshold level comprises:
(a) determining a current amplitude value of the second signal;
(b) determining whether the current amplitude value of the second signal falls within the threshold range; and
(c) repeating steps (a) and (b) while moving the incident energy beam across the scan region the second time.

41.-44. (canceled)

45. A method according to claim 1 wherein determining the threshold level based on the identified extremum comprises determining a threshold range, the threshold range extending above and below the identified extremum and wherein comparing the second signal to the threshold level comprises:
(a) sampling and digitizing the second signal for a peak detect timer period to obtain a second digital signal;
(b) obtaining a maximum value of the second digital signal;
(c) determining whether the maximum value of the second digital signal falls within the threshold range; and
(d) repeating steps (a), (b) and (c) while moving the incident energy beam across the scan region the second time.

46.-50. (canceled)

51. A vein locator comprising:
a transmitter for directing an incident energy beam toward organism tissue, at least a portion of the incident energy beam reflecting from structures within the organism tissue as a reflected energy beam;
a receiver, the receiver connected to detect a first reflected energy beam when the incident energy beam is moved across a scan region a first time and configured to produce a corresponding first signal representative of at least one characteristic of the first reflected energy beam, the receiver connected to detect a second reflected energy beam when the incident energy beam is moved across the scan region a second time and configured to produce a corresponding second signal representative of at least one characteristic of the second reflected energy beam; and
a controller connected to receive the first and second signals from the receiver and configured to:
identify an extremum of the first signal and determine a threshold level based on the identified extremum; and
compare the second signal to the threshold level while the incident energy beam is being moved across the scan region the second time to locate a vein.

52. A vein locator according to claim 51 wherein the transmitter comprises an electromagnetic radiation source and the incident energy beam comprises an incident electromagnetic radiation beam having a wavelength in a range of 600-1,000 nm.

53. (canceled)

54. A vein locator according to claim 52 wherein the transmitter comprises a modulator connected to modulate the incident electromagnetic radiation beam with a carrier frequency in a range of 1-50 kHz.

55. (canceled)

56. A vein locator according to claim 52 comprising one or more moveable optical elements and wherein the controller is connected to controllably move the one or more optical elements to autonomously move the incident electromagnetic radiation beam across the scan region.

57. A vein locator according to claim 52 wherein the controller is connected to controllably move the electromagnetic radiation source to autonomously move the incident electromagnetic radiation beam across the scan region.

58.-60. (canceled)

61. A vein locator according to claim 51 wherein the extremum of the first signal comprises a minimum of the first signal and wherein the controller is configured to identify the minimum of the first signal by:
(a) determining a current amplitude value of the first signal;
(b) comparing the current amplitude value to a minimum value variable (MIN);
(c) if the current amplitude value is less than the minimum value variable (MIN), replacing the minimum value variable (MIN) with the current amplitude value;
(d) repeating procedures (a), (b) and (c) while the incident energy beam is moved across the scan region the first time; and
(e) after the incident energy beam is moved across the scan region the first time, assigning the minimum value variable (MIN) to be the minimum of the first signal.

62. A vein locator according to claim 61 wherein the controller is configured to perform a single repetition of procedures (a), (b) and (c) in a time wherein the incident energy beam is moved less than 0.01 cm across the scan region.

63. (canceled)

64. A vein locator according to claim 54 wherein the extremum of the first signal comprises a minimum of the first signal and the controller is configured to identify the minimum of the first signal by:
(a) sampling and digitizing the first signal for a peak detect timer period to obtain a first digital signal;
(b) obtaining a maximum value of the first digital signal;
(c) comparing the maximum value of the first digital signal to a minimum value variable (MIN);
(d) if the maximum value of the first digital signal is less than the minimum value variable (MIN), replacing the minimum value variable (MIN) with the maximum value of the first digital signal;
(e) repeating procedures (a), (b), (c) and (d) while the incident energy beam is moved across the scan region the first time; and
(f) after the incident energy beam is moved across the scan region the first time, assigning the minimum value variable (MIN) to be the minimum of the first signal.

65. A vein locator according to claim 64 wherein the controller comprises an A/D converter for sampling and digitizing the first signal, and wherein a sampling rate of the A/D converter is 10 or more times the carrier frequency.

66. (canceled)

67. A vein locator according to claim 64 wherein the peak detect timer period is greater than a period of the carrier frequency.

68.-70. (canceled)

71. A vein locator according to claim 51 wherein the threshold level is one or more of: offset from the identified extremum; and scaled from the identified extremum.

72. (canceled)

73. (canceled)

74. A vein locator according to claim 51 wherein the controller is configured to compare the second signal to the threshold level by:
(a) determining a current amplitude value of the second signal;
(b) determining whether the current amplitude value of the second signal is less than the threshold level; and
(c) repeating procedures (a) and (b) while the incident energy beam is moved across the scan region the second time.

75. A vein locator according to claim 74 wherein the controller is configured to perform a single repetition of procedures (a) and (b) in a time wherein the incident energy beam is moved less than 0.01 cm across the scan region.

76. (canceled)

77. A vein locator according to claim 74 wherein the controller is configured activate an indicator to indicate that a current location of the incident energy beam represents a location of the vein, if the current amplitude value of the second signal is determined to be less than the threshold level.

78. A vein locator according to claim 51 wherein the controller is configured to compare the second signal to the threshold level by:
(a) sampling and digitizing the second signal for a peak detect timer period to obtain a second digital signal;
(b) obtaining a maximum value of the second digital signal;
(c) determining whether the maximum value of the second digital signal is less than the threshold level; and
(d) repeating procedures (a), (b) and (c) while the incident energy beam is moved across the scan region the second time.

79.-81. (canceled)

82. A vein locator according to claim 51 wherein the threshold level comprises a threshold range extending above and below the identified extremum and wherein the controller is configured to compare the second signal to the threshold level by:
(a) determining a current amplitude value of the second signal;
(b) determining whether the current amplitude value of the second signal falls within the threshold range; and
(c) repeating procedures (a) and (b) while the incident energy beam is moved across the scan region the second time.

83.-85. (canceled)

86. A vein locator according to claim h 51 wherein the threshold level comprises a threshold range extending above and below the identified extremum and wherein the controller is configured to compare the second signal to the threshold level by:
(a) sampling and digitizing the second signal for a peak detect timer period to obtain a second digital signal;
(b) obtaining a maximum value of the second digital signal;
(c) determining whether the maximum value of the second digital signal falls within the threshold range; and
(d) repeating procedures (a), (b) and (c) while the incident energy beam is moved across the scan region the second time.

87.-91. (canceled)
